(19) United States
(12) Patent Application Publication
(10) Pub. No.: US 2013/0280814 A1
(43) Pub. Date: Oct. 24, 2013

(54) METHOD AND KIT FOR PROTEIN LABELING

(71) Applicant: Elsemarie Bjellqvist, Stockholm (SE)

(72) Inventors: Bengt Bjellqvist, Stockholm (SE); Erik Bjerneld, Uppsala (SE); Ronnie Palmgren, Uppsala (SE)

(73) Assignee: GE HEALTHCARE BIO-SCIENCES AB, UPPSALA (SE)

(21) Appl. No.: 13/699,890

(22) PCT Filed: May 24, 2011
(86) PCT No.: PCT/SE11/50643

§ 371 Date: Apr. 1, 2013

(30) Foreign Application Priority Data
May 27, 2010 (SE) 1050527-9

(51) Int. Cl. C07K 1/13; G01N 33/68

(52) U.S. Cl. CPC C07K 1/13; G01N 33/6839
USPC 436/86; 530/402; 530/409; 530/408; 530/387.1; 530/369

(57) ABSTRACT

The present invention relates to a method for labeling proteins in a sample prior to separation thereof using a protein reactive dye, comprising the following steps a) dissolving the proteins in, or diluting the proteins with, or exchanging an existing protein buffer with, a labeling buffer comprising a dye-reactant (reacting with the protein reactive dye) to form a mixture, b) adding protein reactive dye to said mixture, c) incubating said mixture wherein the labeling of said proteins with said dye can be completed within 5 minutes, and wherein both the proteins and the dye-reactant form measurable reaction products with said dye, and d) separating said reaction products. The invention also relates to a kit for pre-labeling of proteins, comprising a labeling buffer, a dye, a molecular weight marker, and a sample gel loading buffer.

### FIELD OF THE INVENTION

The present invention relates to dye labeling of proteins in samples before separation and analysis thereof. The invention also relates to a labeling kit composed of a labeling buffer, a dye, molecular weight markers, and a sample gel loading buffer.

### BACKGROUND OF THE INVENTION

Labeling proteins with fluorescent dyes has become the method of choice for tracking and quantifying proteins. Fluorescent labeling results in good sensitivity and a broad linear detection range. It also presents a convenient alternative to protein staining methods and is a safer option to radioactive labeling.

The choice of dye and labeling conditions depend on the application. For immunological applications, e.g. antibody labeling, it is important to get high signal intensity and the dye-to-protein ratio is optimized accordingly. For electrophoresis it is also necessary to use a suitable dye-to-protein ratio, in this case to get both high signal intensity and sharp electrophoresis bands. Furthermore, for isoelectric focusing (IEF) electrophoresis it is necessary to use charge-matched dyes to not change the isoelectric point of the protein. Pre-labeling for electrophoresis is well known (see e.g. “Electrophoresis” by Anthony T. Andrews, Clarendon Press, Oxford, 1986).

It is common to label amines in proteins, both the lysine ε-NH₂ groups and the α-NH₂ N-terminal groups. The labeling of proteins using amine reactive fluorescent dyes is usually carried out in buffers free of primary amines. However, the primary amine 2-amino-2-(hydroxymethyl)-1,3-propanediol (Tris) has several attractive features, e.g. low price, non-toxicity, and good buffering capacity at optimal labeling pH, and has therefore been used in low concentrations in the labeling buffer. For example, labeling with dyes for 2D difference gel electrophoresis (DIGE) in 10-40 mM Tris, and in certain cases up to 50 mM, has been recommended from manufacturers.

However, although fluorescent pre-labeling of protein amines has become golden standard for quantitative analysis of proteins in 2D electrophoresis, using the DIGE CyDye™ N-hydroxysuccinimidyl (NHS) esters, traditional Coomassie and silver staining is still widely used for analysis of 1D electrophoresis gels. The lack of commercially available pre-labeling kits for 1D slab gels is partly due to technical limitations of the current labeling methods.

A major technical limitation is that all current labeling protocols are time consuming with many manual steps, e.g. pre-measuring the total protein concentration of the protein, dissolving dye in dimethylformamide (DMF) or dimethyl sulfoxide (DMSO), changing or diluting the sample to obtain low buffer strength, mixing sample with a labeling buffer to obtain optimal pH, adjusting the volumes of dye and protein to a desired protein-to-dye ratio, labeling on ice for 30 minutes, and admixing a stop solution after the labeling. It is much desirable to minimize the time required for labeling.

Another major limitation is that quantification using fluorescence requires comparing the fluorescence signal of a protein of interest to the signal from a reference protein of known amounts. However, in many cases the labeling reaction is highly dependent on the sample content, e.g. sample pH, salts, denaturing agents, reducing agents and detergents. It is also possible that proteins which differ in terms of reactivity and diffusion coefficients compete for a limited amount of dye. In such samples, the signal response from a protein depends on other proteins in the sample which is not desirable. Thus, it is critical that the labeling is robust, i.e. not affected by the sample composition. To obtain robust protocols time-consuming steps are typically added, e.g. to exchange buffer or increase the concentration of proteins, to ensure that the labeling is performed under the same conditions. It is also desirable to minimize sample-to-sample variation caused by differences in pH, temperature, and pipetting errors. In conclusion, there is a great need for a fast and robust labeling protocol, and an internal reaction standard which indicates the labeling efficiency.

### SUMMARY OF THE INVENTION

The present invention provides methods and compositions which solves the technical problems stated above. The present inventors have found that the labeling reaction can be performed in presence of a compound consuming the fluorescent dye (referred to as the dye reactant). By including a dye reactant with appropriate reactivity, at a suitable dye/(dye-reactant) ratio, in the labeling reaction it is possible to control the labeling reaction to obtain a low protein modification level suitable for, for example, electrophoresis. The sample can be any mixture containing proteins. The incubation time of the labeling reaction can be less than 5 minutes and there is no need to pre-measure the protein concentration.

The present invention provides methods and compositions for labeling, separating, and quantitatively analyzing proteins. In particular, the present invention provides a labeling method which is rapid and allows for accurate quantitative analysis after protein separation. The method is especially useful in analytical applications, when high speed and sensitivity is required.

Thus, in a first aspect the invention relates to a method for labeling proteins in a sample prior to separation thereof using a protein reactive dye comprising an amine-reactive, thiol-reactive, or carbonyl-reactive dye, comprising the following steps a) dissolving the proteins in, or diluting the proteins with, or exchanging an existing protein buffer with, a labeling buffer comprising a dye-reactant (reacting with the protein reactive dye) to form a mixture, b) adding protein reactive dye to said mixture, c) incubating said mixture wherein the labeling of said proteins with said dye can be completed within 10 minutes, wherein both the proteins and the dye-reactant form measurable reaction products with said dye, and d) separating said reaction products.

In one embodiment the dye-reactant is provided in excess compared to reactive groups on sample proteins, such as amine, thiol, or carbonyl groups.

The way in which the reaction product is measured depends on the selected protein reactive dye, for example if a fluorescent dye is used, then the fluorescence of the resulting reaction product is measured.

According to a preferred embodiment of the invention, the amount of reaction product from dye and dye-reactant is measured after protein separation and used for correlation of protein signals from proteins labeled in different labeling reactions.

Preferably, the dye-reactant is an amine and is selected from amines such as Tris, 4-amino-1-butanol, 3-amino-1-propanol, 2-amino-1-propanol, 2-amino-2-ethyl-1,3-propanediol, 2-amino-2-methyl-1,3-propanediol, 2-aminoethanol, glycine, lysine, alanine, morpholine, and imidazole. The dye-reactant of the labeling buffer may comprise 50-5000 mM Tris, preferably 200-2000 mM Tris.

In an alternative embodiment, the dye-reactant is an amine-comprising polymer, such as poly-lysine, albumin, aprotinin, or immunoglobulin (IgG).

The protein reactive dye is preferably a fluorescent dye, such as an amine reactive dye. A preferred fluorescent dye is a cyanine dye. In one embodiment for sodium dodecyl sulfate polyacrylamide gel electrophoresis (SDS-PAGE), the cyanine dye comprises one or more sulfonate groups to make the dye water soluble. In an alternative embodiment for SDS-PAGE the dye is charge-matched to not change the protein pI upon conjugation. For IEF electrophoresis the dye is preferably charge-matched to not change the protein pI upon conjugation. The dye may be pre-dispensed in DMF or DMSO.

The dye-reactant may also comprise a functional group enabling separation of the dye-reactant before separation of the labeled proteins.

The labeling reaction may be followed by mixing the labeled sample with a second buffer which is designed for further processing of the sample, e.g. electrophoresis. An example of such a buffer is 125 mM Tris-Cl pH 6.8, 4% (w/v) sodium dodecyl sulfate (SDS), 17% (v/v) Glycerol, 0.1 mg/ml bromophenol blue (BFB), and 200 mM dithiothreitol (DTT).

The labeling buffer may also comprise detergents and is selected from detergents such as sodium dodecyl sulfate (SDS), lithium dodecyl sulfate (LDSI, 3-[(3-Cholamidopropyl)dimethylammonio]-1-propanesulfonate (CHAPS), and nonylphenol ethoxylate (e.g. NP-40). If the labeling buffer comprises anionic detergents the concentration of the detergent is preferably below the critical micelle concentration (cmc).

The labeling buffer may also comprise salt at a concentration up to 2 M, such as NaCl. The labeling buffer may also comprise denaturing agents at concentrations up to 9 M, such as urea and thiourea.

The sample may be pretreated with a reducing agent, such as DTT or tris(2-carboxyethyl)phosphine (TCEP) and optionally an alkylating reagent such as iodoacetamide (IAA), to break protein disulfide bridges prior to labeling.

In a second aspect the invention also relates to a kit for labeling of proteins, comprising a labeling buffer, a dye, a molecular weight marker, and a sample gel loading buffer. The labeling buffer may comprise an amine, e.g. Tris, and optionally a salt, e.g. NaCl, and optionally a detergent, e.g. SDS, and optionally denaturing agents, e.g. urea. The markers are used for molecular weight determination after electrophoresis. The sample loading buffer may contain Tris-Cl, SDS, Glycerol, a tracking dye, and optionally a reducing agent like DTT.

The invention provides a storage-stable kit for labeling proteins in a sample prior to separation of the proteins in the sample, comprising a labeling buffer, a fluorescent dye pre-dispensed in an anhydrous organic solvent such as DMF or DMSO, a molecular weight marker, and a sample gel loading buffer. The kit has a shelf-life of at least 6 months.

In addition to rapid labeling, there are several advantages with the novel approach according to the invention. First, the reactivity of the dye reactant and the ratio dye/dye-reactant is chosen to obtain a desired level of protein modification. At low protein concentrations the ratio dye/dye-reactant controls the level of protein modification. This permits a robust method based on mixing a fixed amount of dye with protein samples of varying concentration down to sub ng/μl concentration levels. Despite a high dye-to-protein ratio in the labeling reaction the competing reaction with the dye-reactant results in a controlled modification of the proteins and we obtain narrow protein bands without band broadening or band shifts, and linear response curves of fluorescence signal versus amount of protein. Thus, if a calibration curve is made for quantitation it is not necessary to pre-measure the protein concentration prior to labeling. Secondly, the reactivity of the dye reactant and the ratio dye/dye-reactant is chosen to minimize protein competition for dye. Thirdly, the dye reactant has physicochemical properties which permit separation and subsequent measurement of the fluorescent signal from the dye-dye reactant complex. This signal serves as an internal control of the labeling reaction, and is compared to the fluorescence signals of the proteins of interest. As a result, the method is insensitive to minor differences in temperature and pH.

The dye reactant may be a compound with a high buffering capacity at optimal pH for labeling. This allows a broader range of sample compositions. The higher buffer capacity permits direct dilution of the sample with the labeling buffer prior to labeling in most cases.

As stated above the protein sample may be diluted with labeling buffer containing a detergent to solubilize and denature proteins, an amine buffer, and optionally a salt, e.g. NaCl. The labeling buffer may also contain a denaturing agent, e.g. urea and/or thiourea. Optionally, the sample protein buffer is exchanged to the labeling buffer. The dye is added to the protein in labeling buffer and the labeling reaction can be completed within 5 minutes at room temperature. A sample loading buffer for electrophoresis is added consisting of: SDS, glycerol, tracking dye, Tris-Cl buffer and optionally DTT to reduce the proteins. This sample loading buffer can be used for both horizontal and vertical gel electrophoresis. The mixture is optionally heated for 3-5 min to fully denature the proteins. The samples are then loaded on the gel. The few steps and fast protocol makes this method a faster alternative compared to post staining techniques.

### BRIEF DESCRIPTION OF THE DRAWINGS

FIG. 1 shows a scan image of Low-Molecular Weight (LMW) marker proteins labeled with Cy™5 using either a 100 mM bicarbonate buffer (A, C) pH 8.5 or a 300 mM Tris-Cl pH 8.5 (B, D) labeling buffer. The labeling was performed in 5 min (A, B) and 30 min (C, D). Both labeling buffers contain 0.1% SDS (w/v).

FIG. 2 shows a comparison of labeling protocols using either a fixed dye-to-protein ratio of 325 pmol per 50 μg protein (A) or a protocol which varies the dye-to-protein ratio (B). The sample was LMW proteins, labeled with Cy5. The labeling was performed in 120 mM Tris-Cl, pH 8.5, with 0.1% SDS (w/v).

FIG. 3 shows how the Tris buffer reduces protein competition for dye in the labeling reaction. This was observed by adding lactoglobulin (LG) to bovine serum albumin (BSA) samples and measuring the decrease in BSA Cy5 signal after labeling, using either 30 or 300 mM Tris in the labeling buffer.

FIG. 4 shows a Cy5-prelabeled HeLa UV irradiated cell lysate (˜24 μg) run on a 12% Tris-Glycine gel (A). The cell lysate was labeled in 300 mM Tris-Cl pH 8.5 and 0.2 M NaCl, and 0.1% SDS (w/v). The sample was then transferred to a polyvinylidene fluoride (PVDF) membrane and a western blot analysis was performed. During probing the primary antibody p-ERK monoclonal Ab from mouse was applied, followed by a Cy™3 goat-α-mouse antibody (secondary Ab). The PVDF membrane was scanned in Cy5 (B) and in Cy3 (C). The cell lysate is seen in the Cy5 image and the probed phosphorylated protein is detected in the Cy3 image.

FIG. 5 and FIG. 6 show that the Cy5 NHS ester (PA15101 GE Healthcare) is stable in DMSO and DMF over an 8-month time-period, both for freezer and refrigerator storage. The reference sample was freshly prepared in DMSO, and the labeling efficiency was evaluated using the same amount of dye and protein in the labeling reaction. The LMW markers were labeled in 300 mM Tris-Cl pH 8.5, 0.2 M NaCl, and 0.1% SDS (w/v). The average Cy5 signal intensity of the triplicates (in FIG. 6) for each protein in the Low-Molecular Weight Marker, show a slight reduction in labeling efficiency for refrigerator storage (4° C. to 8° C.) but no reduction in efficiency for freezer (−20° C.) storage compared to the reference sample.

### DETAILED DESCRIPTION OF THE INVENTION

Some labeling methods require determining the total protein concentration prior to labeling. The rationale for this step is that too low protein and dye concentrations lead to poor labeling and low signal-to-noise ratios. A dye-to-protein ratio should be chosen so that the fraction of protein residues reacted with fluorescent dye should be as high as possible, for highest possible sensitivity, without resulting in band broadening or extra bands. However, the pre-determination of protein concentration is not only time and work consuming, the presently used methods for determination of protein concentration (2D Quant Kit, Bradford, UV and biouret based method) does not correlate to the amine content of the sample. The amino acid composition of a sample can vary within wide limits depending on the composition of the major proteins present in the sample. Based on SwissProt statistics an average protein contain 5.2% lysine residues, but the high mobility proteins 1 and 4 (HMG-1 and HMG-4) contain 20.1% lysine, while the lysine content of pepsin A and pepsinogen C is zero or close to zero. Dosing the amount of CyDye NHS ester based on protein concentrations determined with UV, Bradford or biuret based methods will not give a reasonable control of the fraction of the amine residues which will bind dye. The signal intensity resulting from a specific protein can vary within wide limits with varying sample composition. The result is very frequently a non optimal relation between the amine content of the sample and added amount of CyDye NHS ester. Thus, it is desirable to remove the measurement of protein concentration prior to labeling.

The method of the invention eliminates the need to determine the protein concentration prior to labeling, and minimizes protein competition for dye, which result in a more robust and reproducible method compared to labeling methods based solely on fixation of the dye-to-protein ratio. The method constitutes an ideal approach when the concentrations of similar or identical proteins present in sample of different compositions should be compared.

In another embodiment, the concentration of available lysines for labeling is determined prior to labeling, for example using fluorescamine or TNBS (2,4,6,-trinitrobenzene sulfonate) assays, and the amount of dye adjusted if needed.

Following labeling, the labeled proteins and/or protein fragments are separated using techniques including, but not limited to, electrophoresis, chromatography, immuno-assays, and mass spectrometry. The fluorescence signal is detected by fluorescence scanners or imagers (e.g. Typhoon™ scanners from GE Healthcare) providing a broad dynamic range of up to 10⁵.

Another embodiment provides kits and methods with significantly reduced times from labeling to loading the samples on a gel for electrophoresis. This is in part achieved by reducing the labeling reaction time from 30 min on ice to 30 seconds at room temperature. The addition of a dye-reactant eliminates the need to use traditional stop solutions. Instead the labeled sample is mixed with sample loading buffer directly after labeling. The kit also provides the user with pre-dispensed dye to be directly mixed with the protein sample in the labeling buffer. Fluorescent dyes are traditionally packaged and sold dry and the user adds an organic solvent prior to labeling. However, we have found that the dye can be stored cold in anhydrous DMSO, or DMF, for extended periods of time with full labeling activity. This omits the step of having to reconstitute the dye in DMSO or DMF prior to labeling. DMSO is preferably used because it is a non-toxic organic solvent.

In another embodiment, the reaction of a protein sample with CyDye NHS ester in presence of an excess of an amine containing compound consuming a major fraction of the dye can also be used as a very fast and sensitive determination of the protein content of the sample. What is required is a fast and simple way to separate the CyDye tagged protein from the compound resulting from the reaction between the added amine and the dye NHS ester. A simple solution is to use a primary amine containing functional groups that can be used for capture at a column or similar. This method should be ideal for protein determination prior to DIGE experiments as it measures the content of reactive lysine in the sample.

In another embodiment, the proteins are labeled with an amine reactive fluorescent dye. A dye reactant with a reactive amino group is added to the labeling mixture. The reactant is subsequently separated from the proteins, and the dye-reactant complex is detected. The intensity of the fluorescence signal is compared to the signals of the proteins. The dye reactant could be, but is not limited to, a protein, e.g. aprotinin, or an amine, e.g. Tris.

In one embodiment, labeling for SDS-PAGE can be performed with water soluble sulfonated dyes. Thus, the dye can be dosed in dry form, or in an organic solvent, and the customer does not need to add DMF or DMSO to reconstitute the dye prior to labeling.

In another embodiment, charge-matched dyes are used to label proteins without altering the pI of the protein for both IEF electrophoresis and SDS-PAGE.

In another embodiment, the proteins are labeled with an amine reactive fluorescent dye. A dye reactant with a reactive amino group is added to the labeling mixture. The reactant is a buffering compound and maintains a pH in the interval 7-11 in the labeling mixture. In a further embodiment the reactant maintains a pH in the interval 8-9.

In another embodiment for SDS electrophoresis, the dye reactant has, besides an amino group with suitable reactivity, positively charged groups in order to secure that the reactant, as well as the product formed in the reaction between reactant and dye, are transported towards the cathode after sample application. This combination should also be suitable prior to IEF applications where basic application is used.

In another embodiment for SDS electrophoresis, dyes containing sulfonate groups will give a negatively charged reaction product and the dye-reactant product will be transported in the front towards the anode which permit measurement of the dye-reactant signal or allow the dye-reactant to leave the gel prior to scanning.

In another embodiment for IEF electrophoresis, charge-matched dyes are used to label proteins without altering the pI of the protein. The sample is applied at the anodic side in order to avoid disturbances, and the dye-reactant product should be transported towards the anode. To secure this the dye reactant need to contain the reactive amino group and a minimum of two acidic groups. Two possible examples are aspartic and glutamic acid.

In one embodiment, the labeling buffer comprises NaCl to minimize sample salt effects on the labeling reaction. We have found that salt ions may affect the labeling reaction. For example, the addition of NaCl to the labeling buffer increases the Cy5 signal per gram for some proteins using a sulfonated, negatively charged, mono-reactive Cy5 NHS ester. We have also found that NaCl can be added up to 0.5 M in the labeling buffer without affecting the subsequent electrophoresis. Including NaCl in the labeling buffer will in some cases make the labeling more robust, i.e. insensitive to the initial salt concentration of the sample upon dilution with the labeling buffer.

The term “dye reactant” as used herein refers to a chemical compound capable of forming a covalent bond with a fluorescent dye. The chemicals used are selected so that neither the excess of reactant consuming CyDye NHS ester nor the product resulting from the reaction between reactant and NHS ester disturb the electrophoretic separation.

### EXPERIMENTAL SECTION

### Material:

Cy5: Stock solutions of Cy5 were prepared by dissolving mono-reactive Cy5 NHS ester (PA15101 GE Healthcare) or the Cy5-DIGE NHS ester (258010-85 GE Healthcare) in either anhydrous DMF (227056 Sigma) or anhydrous DMSO (276855 Sigma) at concentrations ranging from 0.1-1 mg/ml.

Samples: Low-molecular weight (LMW) marker protein Kit (17-0446-01 GE Healthcare), lactalbumin (L5385 Sigma Aldrich) and bovine serum albumin (A7638 Sigma-Aldrich), were either dissolved in phosphate buffered saline (PBS) buffer, or in the labelling buffer directly. HeLa lysate (SC 2221) and p-ERK (SC 7383) were from Santa Cruz Biotechnology.

2× Sample Loading Buffer: a solution of 0.125 M Tris-Cl pH 6.8, 4% (w/v) SDS, 17.4% (v/v) glycerol, 0.1 mg/ml BFB, and 0.2 M DTT was used. The chemicals tris, glycerol, SDS, BFB, and DTT were from GE Healthcare.

Electrophoresis: SDS-PAGE gels PhastGel™, ExcelGel™ and Genegel were run on Multiphor™, PhastSystem™ and GenePhor electrophoresis units according to instructions. The 12% tris-glycine gel (EC60055Box from Invitrogen, Life Technologies) was run on a MiniVE vertical electrophoresis system according to instructions. The gels were scanned using Typhoon™ scanners and in some cases subsequently post-stained with Coomassie for comparisons.

Western Blotting. ATE 22 Mini tank transfer unit and Amersham™ Hybond™ blotting paper were used for Western blotting according to instructions.

### Labeling Protocol:

1. Dissolve the protein in, or dilute the protein with, labeling buffer.
2. Add dye to the protein mix. Incubate for a time between 30 seconds and 10 minutes. When labeling several samples in parallel use a time interval of 5-10 minutes.
3. Mix the sample with 2× sample loading buffer in equal volumes
4. Heat the sample for 3-5 min at 95° C. (optional)
5. Apply the sample on the electrophoresis gel

If needed, the buffer of the protein sample can be exchanged to the labeling buffer prior to labeling, e.g. using gel filtration or dialysis. The sample is optionally treated with iodoacetamide (IAA) after step 4 to minimize bandbroadening as a result of protein re-oxidation before or during electrophoresis.

### Example 1

### Tris as Dye Reactant

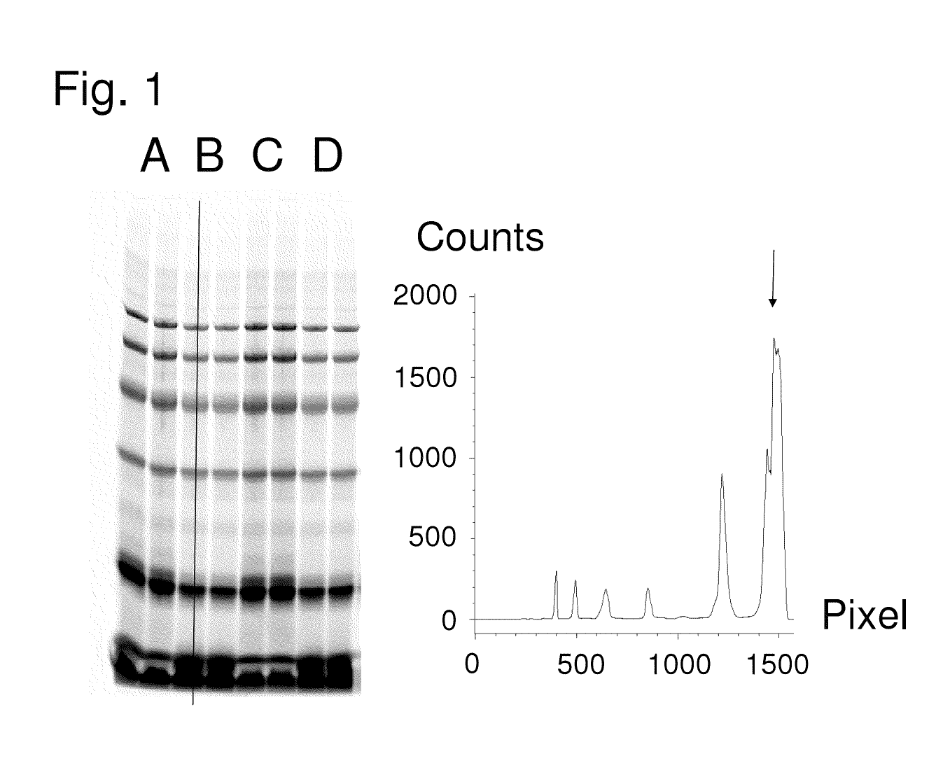

The present inventors have found that Tris can be used at high concentrations in the labeling buffer, see FIG. 1-6. The use of Tris in the labeling buffer compared to an amine free buffer like bicarbonate, results in a decrease in protein Cy5-signal and a measurable signal from a Cy5-Tris complex near the electrophoresis front, indicated by the arrow in FIG. 1. The signal of the non-protein bound Cy5 can thus be used as an internal standard of the labeling reaction. The low-molecular weight marker proteins were labeled in 100 mM bicarbonate buffer (A,C) pH 8.5 and a 300 mM Tris-Cl pH 8.5 buffer (B, D) for 5 min (A,B) and 30 min (C,D). Both buffers contained 0.1% SDS (w/v). The proteins were subsequently mixed with sample loading buffer and separated on an electrophoresis gel. The Cy5 scan image shows band patterns which are very similar for both buffers. There is no difference in Cy5 signal patterns for 5 min versus 30 min showing that the labeling reaction is complete within 5 min.

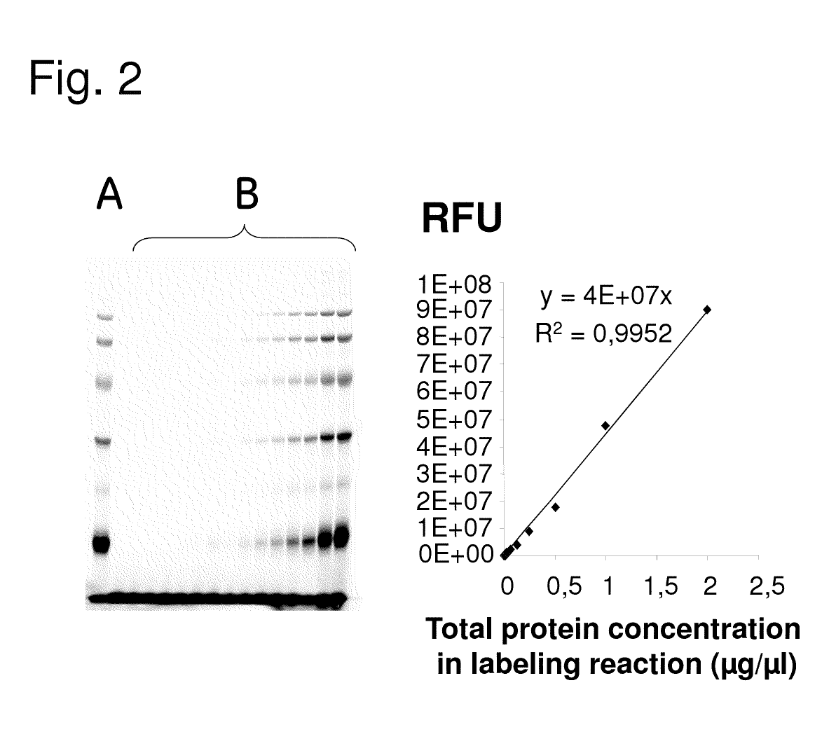

FIG. 2 shows a comparison of two labeling protocols using a fixed dye-to-protein ratio of 325 pmol per 50 μg protein (A) and a protocol which varies the dye-to-protein ratio (B). The labeling was performed in 120 mM Tris-Cl pH 8.5 with 0.1% (w/v) SDS using a 5 min labeling time. For the B-series, the protein concentration in the labeling reaction was varied from 0.1 ng/μl to 2.0 μg/μl, a fixed amount of 325 pmol dye was used in a reaction volume of 10 μl. Despite the high dye-to-protein ratio, it was possible to label proteins with a controlled protein modification level. This is evidenced by the fact that there is no bandbroadening or detectable shifts in positions on the gel despite the high theoretical dye-to-protein ratios, which shows that hydrolysis of the dye and/or a side-reaction with Tris and the dye compete with the protein labeling reaction. Furthermore, the labeling protocol allows for accurate quantification using a calibration curve. FIG. 2 shows the linear lactalbumin Cy5 signal versus concentration of total protein in the labeling reaction. The ratio of the amount of lactalbumin to the total protein amount (in weight) was constant, approximately ⅕. Thus, this protocol removes the need for a pre-determination of protein concentration. The detection limit, using the 12.5% polyacrylamide gel and a sample loading volume on the gel of 6 μl, was sub-ng.

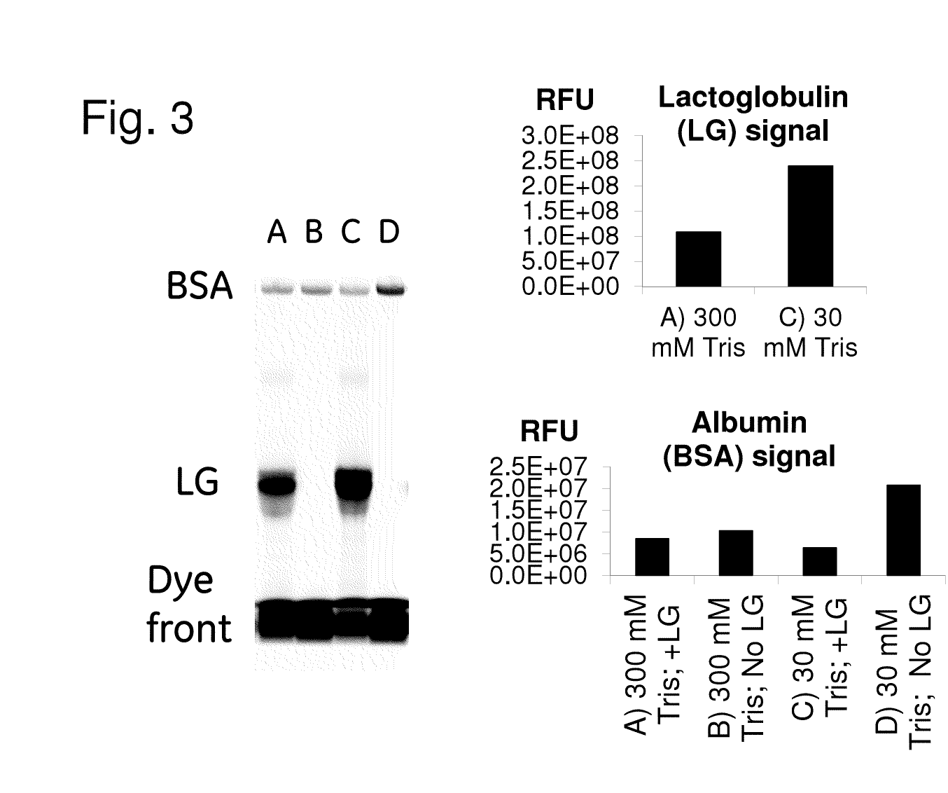

It is possible that proteins compete for dye in the labeling reaction. However, a high Tris concentration decreases the protein competition for dye. FIG. 3 shows protein competition for dye observed by adding lactoglobulin (LG) to bovine serum albumin (BSA) samples and measuring the decrease in BSA Cy5 signal after labeling. The amount of Cy5 and BSA was kept constant in the labeling series and LG was added in excess to BSA (9 times the amount in weight). FIG. 1 (left) shows a Cy5 scan image of four samples labeled in 300 mM (lane A and B) and in 30 mM (lane C and D) Tris-Cl pH 8.5. The 300 mM Tris labeling buffer significantly reduces the protein competition and gives more accurate LG/BSA ratios (˜10). Triplicate samples were run on both PhastGels 8-25% and GeneGels 12.5%, the relative standard deviation of the Coomassie signals from BSA (N=24) was 8%. This experiment shows that protein competition for Cy5 dye decreases if a high concentration of Tris is used. The decrease in protein competition was observed using protein concentrations between 0.1-1 μg/μl and Cy5 amounts in the range 0.5-5 nmol in the labeling reaction, and a reaction volume of 80 μl.

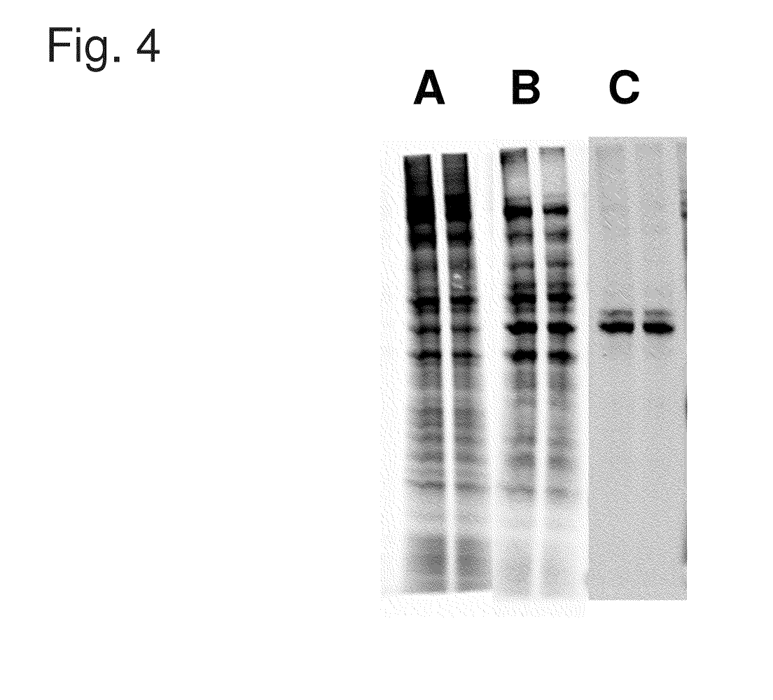

The high Tris concentration in the labeling buffer is also ideal when labeling complex samples, e.g. cell lysates. The high buffering capacity of the labeling buffer (300 mM Tris-Cl pH 8.5 and 0.2 M NaCl, and 0.1% w/v SDS) allow for easy mixing of sample and labeling buffer prior to labeling. FIG. 4 shows the results of the efficient and fast labeling of a HeLa lysate, which allow for quantitative detection of both the target protein p-ERK and the total protein content on the membrane using a fluorescence scanner.

### Example 2

### 3-Amino-1-Propanol as Dye Reactant

In this example, 3-amino-1-propanol was used as dye-reactant at 1-300 mM concentrations in the labeling reaction. The labeled proteins, beta-lactoglobulin, bovine serum albumin, and bovine carbonic anhydrase, were all detected with good signal-to-noise ratios. The reaction product between the dye-reactant amine and CyDye NHS ester was negatively charged and transported towards the anode during SDS-PAGE.

Furthermore, a number of different small amines were tested, including 2-amino-2-methyl-1,3-propanediol, 2-amino-2-ethyl-1,3-propanediol, 2-aminoethanol, and morpholine, in the labeling reaction which resulted in detectable signals from both proteins and the reaction product of dye and amine.

### Example 3

### Amylase Protein as Dye-Reactant

In this example, the protein amylase was present in the labeling reaction in excess compared to the target LMW proteins. The amounts of amylase and Cy5 in the labeling reaction were kept constant and the amount of LMW proteins was varied. Both the LMW proteins and amylase could be easily detected and there was no disturbing bandbroadening of the LMW proteins. The signal of the amylase protein could be used to correlate the signal from target proteins labeled in different labeling reactions.

### Example 4

### Pre-Labeling Kit with a Pre-Dispensed Dye in DMSO

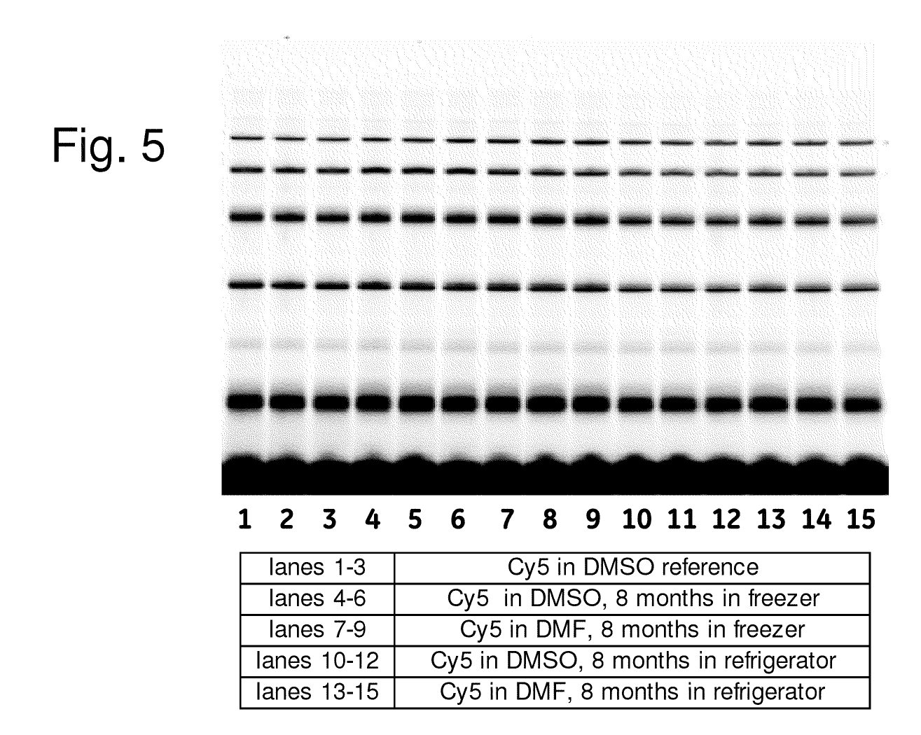

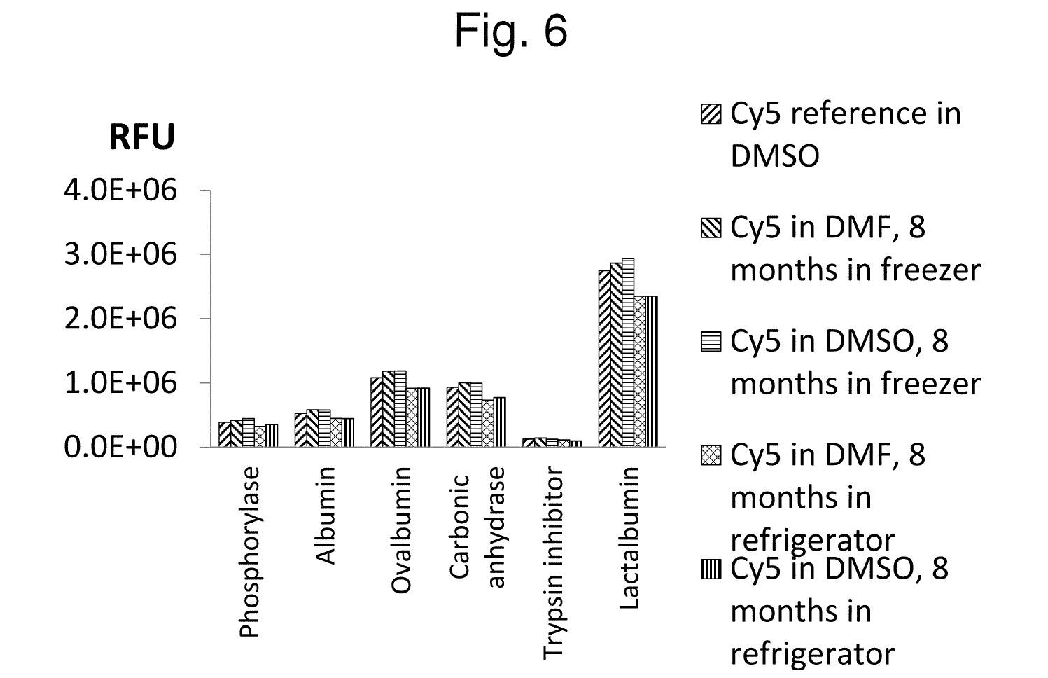

The inventors have found that the Cy5 NHS ester is very stable when dissolved in anhydrous organic solvents, e.g. DMSO, and stored cold in the freezer. FIG. 5 and FIG. 6 show that the Cy5 NHS ester can be stored for over 8 months in the freezer (at −20° C.) and still exhibit full labeling efficiency. This finding allow for novel formulations of the dye in protein labeling kits.

These examples show that fast labeling can be carried out using high concentrations of dye-reactants, in these examples reactive amines, in the labeling buffer. There are several important advantages that can be gained using a high concentration of amines in the labeling buffer, including
easy dilution of sample to obtain an optimal pH for labeling as a result of better buffer capacity
accurate quantification of proteins as a result of less protein competition for dye
not necessary to use a separate stop solution with a dye-reactant after the labeling reaction
no need for changing buffer after labeling as a result of better conductivity matching to the gel buffer, which is usually 375 mM Tris.

Furthermore, the inventors have found a labeling protocol which eliminates the need for pre-measuring the total protein concentration prior to labeling. Using a fixed amount of dye per reaction, and dye-reactants to control the amount of available dye for protein labeling, it is possible to obtain narrow bands and no shifts in band position, and linear response curves (signal versus amount of protein) for a wide range of protein concentrations (sub ng/μl to μ/μl) in the sample.

The invention also relates to a novel kit for pre-labeling proteins prior to electrophoresis, comprising a labeling buffer with a dye-reactant, a storage-stable dye, a molecular weight marker, and a sample gel loading buffer.

1. A method for labeling proteins in a sample prior to separation thereof using a protein reactive dye, comprising the following steps a) dissolving the proteins in, or diluting the proteins with, or exchanging an existing protein buffer with, a labeling buffer comprising a dye-reactant (reacting with the protein reactive dye) to form a mixture, b) adding protein reactive dye to said mixture, c) incubating said mixture wherein the labeling of said proteins with said dye can be completed within 10 minutes, and wherein both the proteins and the dye-reactant form measurable reaction products with said dye, and d) separating said reaction products.

2. The method of claim 1, wherein the labeling of proteins is completed within 5 minutes.

3. The method of claim 1, wherein the labeling of proteins is completed within 30 seconds.

4. The method of claim 1, wherein the dye-reactant is provided in excess compared to the reactive groups on sample proteins, such as amine, thiol, or carbonyl groups.

5. The method of claim 1, wherein the amount of reaction product from dye and dye-reactant is measured after protein separation and used for correlation of protein signals from proteins labeled in different labeling reactions.

6. The method of claim 1, wherein the dye-reactant is an amine and is selected from amines such as Tris, 2-amino-2-methyl-1,3-propanediol, 2-amino-1-propanol, 2-amino-2-ethyl-1,3-propanediol, 4-amino-1-butanol, 3-amino-1-propanol, 2-aminoethanol, glycine, lysine, poly-lysine, alanine, morpholine, and imidazole.

7. The method of claim 1, wherein the dye-reactant is Tris and the labeling buffer comprises 50-5000 mM Tris, preferably 200-2000 mM Tris.

8. The method of claim 1, wherein the protein reactive dye is a fluorescent dye, such as a cyanine dye.

9. The method of claim 8, wherein the dye is a cyanine dye comprising sulfonate groups to make the dye water soluble.

10. The method of claim 8, wherein the dye is charge-matched to not change the protein pI upon conjugation.

11. The method of claim 1, wherein the dye is dispensed in DMF or DMSO, and a fixed amount of dye is used per labeling reaction.

12. The method of claim 1, wherein the dye-reactant is an amine-comprising protein other than the proteins in the sample, such as albumin, aprotinin or IgG.

13. The method of claim 1, wherein the dye-reactant also is provided with a functional group enabling separation of the dye-reactant before separation of the labeled proteins.

14. The method of claim 1, wherein the labeling reaction is followed by mixing the labeled sample with a second buffer which is designed for further processing of the sample, e.g. electrophoresis.

15. The method of claim 1, wherein the labeling buffer also comprises detergents and is selected from detergents such as SDS, lithium dodecyl sulfate (LDS), 3-[(3-Cholamidopropyl)dimethylammonio]-1-propanesulfonate (CHAPS), and nonylphenol ethoxylates.

16. The method of claim 1, wherein the labeling buffer also comprises anionic detergents at sub-cmc concentrations, such as the detergents SDS and LDS, and/or a salt at a concentration up to 2 M, such as NaCl.

17. The method of claim 1, wherein the labeling buffer also comprises denaturing agents, such as urea and thiourea at concentrations up to 9 M.

18. The method of claim 1, wherein the sample is pretreated with a reducing agent, such as DTT or tris(2-carboxyethyl)phosphine (TCEP), and optionally an alkylating reagent such as IAA, to break protein disulfide bridges prior to labeling.

19. A kit for labeling proteins in a sample prior to separation of the proteins in the sample, comprising a labeling buffer with a dye-reactant, a protein-reactive dye, a molecular weight marker, and a sample gel loading buffer.

20. The kit of claim 19, wherein the dye is a storage-stable fluorescent dye pre-dispensed in an anhydrous organic solvent such as DMF or DMSO.

21. The kit of claim 19, wherein the dye is water-soluble and pre-dispensed in dry form.

22. The kit of claim 19, wherein the dye does not change the pI of the protein upon labeling.

23. The kit of claim 19, wherein the labeling buffer comprise a dye-reactant at high concentration, such as Tris at a concentration of 200-2000 mM.

24. The kit of claim 19, wherein the labeling buffer comprise a protein dye-reactant which is different from the protein to be labeled, such as albumin or aprotinin.

25. The kit of claim 19, wherein the labeling buffer and sample loading buffer replace a separate stop solution after labeling.
